(19) United States
(12) Patent Application Publication
(10) Pub. No.: US 2011/0236494 A1
(43) Pub. Date: Sep. 29, 2011

(54) CIPROFLOXACIN ORAL SUSPENSION

(75) Inventors: Sumit Kumar SAHA, Purnea (IN); Mona DHALIWAL, Saharanpur (IN); Swati AGGRAWAL, New Delhi (IN); Mukesh Kumar GARG, Ludhiana (IN); Ajay Kumar SINGLA, Chandigarh (IN)

(73) Assignee: RANBAXY LABORATORIES LIMITED, Delhi (IN)

(21) Appl. No.: 13/035,200

(22) Filed: Feb. 25, 2011

(30) Foreign Application Priority Data
Mar. 4, 2010 (IN) 486/DEL/2010

(51) Int. Cl. A61K 9/16; A61K 31/496; A61P 31/04; B05D 1/02

(52) U.S. Cl. 424/492; 424/490; 514/253.08; 424/494; 424/497; 424/496; 427/2.14

(57) ABSTRACT

The present invention relates to oral taste masked pharmaceutical composition comprising ciprofloxacin or salts or esters thereof. It further relates to processes of preparing it.

### FIELD OF THE INVENTION

The present invention relates to oral taste masked pharmaceutical compositions comprising ciprofloxacin or salts or esters thereof and processes for their preparation.

### BACKGROUND OF THE INVENTION

Oral administration is the preferred route of administration for pharmaceutical compositions. But such compositions are associated with certain disadvantages, particularly in the treatment of patients having dysphagia, i.e., who have difficulty in swallowing, thereby leading to patient incompliance.

Liquid dosage forms represent a viable alternative, but usually lead to direct exposure of the active drug ingredient to taste buds and this is a serious problem when the drug has an extremely unpleasant or bitter taste. Taste is an important parameter governing patient compliance. Most pharmaceutical actives are unpleasant tasting and that taste can range from a lingering chemical taste to a harsh bitterness, with intensities varying from moderate to high.

Ciprofloxacin is a fluroquinolone antibiotic having an extremely unpleasant taste. Many methods have been disclosed in the prior art to achieve the taste-masking effect, such as the use of ion-exchange resins, salt and ester conjugation, lipids, solid dispersions and microencapsulation, but none have provided a complete concealment of taste together with a rapid release of the drug.

U.S. Pat. No. 7,175,856 describes a pharmaceutical formulation in the form of a palatable oral suspension. The particles of the drug are rendered and maintained in a substantially insoluble form through use of one or more pH modifying agents, which are mixed with particles of the drug. Thus, when the drug and pH-modifying agent are mixed with water for reconstitution into a suspension, the pH-modifying agent adjusts suspension pH to reduce or minimize solubility of the drug and reduces or masks the bitter taste normally associated therewith.

U.S. Pat. No. 6,767,557 provides a taste masked pharmaceutical composition that includes a microcapsule, wherein the microcapsule includes a pharmaceutically active agent core coated with a taste-masking effective amount of a water-insoluble enteric coating.

EP Patent Application No. 378,137 describes water-dispersible pharmaceutical preparations that make it possible to administer orally active ingredients having organoleptically unfavorable properties in liquid form. The active ingredient is first applied to sugar spherules, which subsequently are provided with a film layer. A combination of water insoluble polymers and polymers soluble below pH 5 is used for coating the drug layered cores.

U.S. Pat. No. 5,695,784 discloses taste-masked microcapsules that include a highly bitter drug, like ciprofloxacin. The active ingredient is present as an anhydrate or its base form and the microparticles are coated with a combination of water-soluble and water-insoluble polymers. The patent describes that the rapid release of the active ingredient from the microcapsules is achieved only when it is present as an anhydrate of its base form. Further this patent also discloses that drug layering on inert cores like sugar spherules does not provide a complete concealment of taste and rapid release for a highly bitter and high dose drug like ciprofloxacin.

The present inventors have now developed a taste-masked formulation for ciprofloxacin that includes an inert core layered with ciprofloxacin or salts or esters thereof and a taste-masking coating. The formulation of the present invention provides both a complete taste-masking effect and a rapid release of the drug.

### SUMMARY OF THE INVENTION

In one general aspect, the present invention provides for a taste-masked pharmaceutical composition, which includes a microgranular core of ciprofloxacin and a taste-masking coating.

Embodiments of this aspect may include one or more of the following features. For example, the ciprofloxacin is present as the freebase form. The microgranular core of ciprofloxacin is an inert core layered with ciprofloxacin. The inert core may be pharmaceutically acceptable inert insoluble materials, soluble materials, or commercially available products. The insoluble inert cores may be one or more of dicalcium phosphate, or microcrystalline cellulose. The soluble inert cores may be one or more of glucose, mannitol, lactose, xylitol, dextrose, sucrose and mixtures thereof. The commercially available inert cores may be one or more of sugar spheres, non-pareil seeds, celpheres and mixtures thereof.

The taste masking coating includes a mixture of water insoluble and water soluble polymers. The water insoluble polymer may be one or more of acacia gum, acrylic acid polymers and copolymers (polyacrylamides, polyacryldextrans, polyalkyl cyanoacrylates, polymethyl methacrylates), agar-agar, agarose, albumin, alginic acid and alginates, carboxyvinyl polymers, cellulose derivatives, such as, cellulose acetate, polyamides (nylon 6-10, poly(adipyl-L-lysines, polyterephthalamides and poly(terephthaloyl-L-lysines)), poly-caprolactam, polydimethylsiloxane, polyesters, poly(ethylene-vinyl acetate), polyglycolic acid, polylactic acid and its copolymers, polyglutamic acid, polylysine, polystyrene, shellac, xanthan gum, anionic polymers of methacrylic acid and methacrylic acid esters.

The water soluble polymer may be one or more of hydroxypropylcellulose, hydroxypropylmethylcellulose, methylcellulose, sodium carboxymethylcellulose, dextran, dextrins, cyclodextrins, polyethylene glycols, polyvinyl alcohols, polyvinylpyrrolidones, starch and starch-hydrolysates, for example, modified types of starch (gelatinized starch, celutab, maltodextrins), sugars and sugar replacements, such as, mono-, di- and oligosaccharides, sucrose, fructose, lactose, dextrose, mannitol, sorbitol and xylitol and alginic acid and alginates, tragacanth, pectins, gum arabic and gelatin.

The taste masking coating may be a mixture of neutral methyl ester and/or ethyl ester compounds of polymethacrylic acid and hydroxypropylmethylcellulose. The taste masking coating layer may further include one or more pharmaceutically acceptable inert excipients.

The one or more pharmaceutically acceptable inert excipients include plasticizers, lubricants, wetting agents, or colorants.

The plasticizers include one or more of diethyl phthalate, acetyl tributylcitrate, glycerol, diethyl sebacate, dimethyl phthalate, dibutyl phthalate, tributyl citrate, butyl stearate, polyethylene glycols of different chain lengths, glycerol monostearate, triacetin, castor oil and other native and synthetic oils, triethyl citrate, acetyl triethylcitrate, 1,2-propylene glycol, acetylated fatty acid glycerides and polyoxyethylene-polyoxypropylene copolymers.

The wetting agents include one or more of sodium lauryl sulphate (USP), polysorbate (20, 40, 60, 80, 65, 61, 85 and 21), poloxamers (ethylene oxide propylene oxide block copolymers) of differing HLBs, lecithins, oleic acid and oleic acid salts, sorbitan esters (Span 20, 40, 60, 80 and 85), propylene glycol monostearate and monolaurate, glycerol monostearate and monooleate, Brij types (fatty alcohol-PEG ethers) of differing HLBs (for example, PEG 10 cetyl ether, PEG 20 oleyl ether etc.), Myrj types (fatty acid-PEG esters) of differing HLBs (for example PEG 40 monostearate; PEG 100 monostearate and the like), sodium dodecylsulphate (SDS), dioctyl sodium sulphosuccinate (DOSS), ethoxylated mono- and diglycerides of differing HLBs (Tagat types), sucrose fatty acid esters, fatty acid salts (Na, K, Ca, Mg, Al etc.), ethoxylated triglycerides (polyoxyethylated castor oil (40), polyoxyethylated hydrogenated castor oil (40 and 60), polyoxyethylated vegetable oils), sterols (cholesterol and wool wax alcohols).

The lubricants include one or more of magnesium stearate, calcium stearate, calcium behenate, talc, colloidal silicic acid, stearic acid, precirol (mixture of mono-, di- and triesters of palmitic and stearic acid with glycerol), hydrogenated cottonseed oil, hydrogenated castor oil and polyethylene glycol of differing molecular weights.

The composition provides a rapid release of ciprofloxacin at pH 1 and 4.5. The composition includes more than 5% w/w of water.

In another general aspect, the present invention provides for a process for making a taste-masked pharmaceutical composition. The process includes:

(i) dispersing ciprofloxacin and binder in a solvent to form a drug-dispersion;

(ii) spraying the dispersion of step (i) over inert cores;

(iii) dispersing water-insoluble and water-soluble coating agents, and optionally a surfactant, in a solvent to form a coating dispersion;

(iv) coating the cores of step (ii) with the coating dispersion of step (iii) to obtain microgranular cores.

### DETAILED DESCRIPTION OF THE INVENTION

The present invention relates to a taste-masked pharmaceutical composition that includes ciprofloxacin or salts or esters thereof and processes for preparing it.

The term “taste-masked”, as used herein, refers to any substance or particle, or oral pharmaceutical composition with an unpleasant tasting pharmaceutically active substance that has been treated to render it palatable and/or which does not substantially release the pharmaceutically active substance in the mouth, but rather for example, in the stomach or the intestinal tract.

Ciprofloxacin used in the composition of the present invention may be present in freebase or salt or ester form, preferably the freebase form is used. Further, the composition of the present invention may include anhydrate or hydrate form of ciprofloxacin. Particularly, ciprofloxacin may be present in the hydrated form.

The taste-masked pharmaceutical composition of the present invention may include more than 5% w/w of water in the form of water of crystallization or other water adducts; thereby excluding the step of drying the microgranules or microcapsules to achieve anhydrous ciprofloxacin. Despite of more than 5% w/w water in the final microcapsules the present invention yields a product which releases ciprofloxacin in pH 1 and pH 4.5 acetate buffer.

The LOD (Loss on Drying) values of the final microcapsules of the present invention determined under the vacuum conditions at 120° C. for 1 hour fall within the range of 6% to 8% w/w. The term “LOD” or Loss on Drying refers to the loss in weight or mass at the specified conditions and is expressed as % w/w or % m/m.

The microgranular core of ciprofloxacin includes an inert core layered with ciprofloxacin. The inert core may be selected from pharmaceutically acceptable inert insoluble or soluble materials. Alternatively the inert core may also be a commercially available product. The insoluble inert cores may be composed of dicalcium phosphate, microcrystalline cellulose and the like, either alone or in combination. The soluble inert cores may be composed of sugar selected from glucose, mannitol, lactose, xylitol, dextrose, sucrose and mixtures thereof. Commercially available inert cores may be sugar spheres, non-pareil seeds, celpheres and mixtures thereof. The cores may be of any geometric shape, although spheres are preferred for the ease of uniform coating.

Ciprofloxacin, with one or more pharmaceutically acceptable excipients, is layered over the inert cores as a powder or as suspension or solution in a suitable solvent.

The microgranular cores of ciprofloxacin may be subcoated to protect the core from the taste-masking coating layer. Suitable materials for the optional sub-coat layer include sugar, polyethylene glycol, polyvinyl pyrrolidone, polyvinyl alcohol, polyvinyl acetate, hydroxypropyl cellulose, methylcellulose, ethylcellulose, hydroxypropyl methylcellulose, carboxymethylcellulose sodium and others, used alone or in mixtures. Additives, such as, plasticizers, colorants, pigments, fillers, anti-tacking and anti-static agents, such as, for instance, magnesium stearate, titanium dioxide, talc, pH-buffering substances and other additives may also be included into the subcoating layer.

The taste-masking coating of the present invention includes a mixture of water-insoluble and water-soluble coating agents to provide effective taste-masking effects together with the rapid release of the drug at the acidic pH level of the stomach.

The taste-masking coating layer may further include one or more pharmaceutically inert excipients like plasticizers, lubricants, wetting agents or colorants.

Plasticizers include diethyl phthalate, acetyl tributylcitrate, glycerol, diethyl sebacate, dimethyl phthalate, dibutyl phthalate, tributyl citrate, butyl stearate, polyethylene glycols of different chain lengths, glycerol monostearate, triacetin, castor oil and other native and synthetic oils, triethyl citrate, acetyl triethylcitrate, 1,2-propylene glycol, acetylated fatty acid glycerides and polyoxyethylene-polyoxypropylene copolymers.

Wetting agents include sodium lauryl sulphate (USP), polysorbate (20, 40, 60, 80, 65, 61, 85 and 21), poloxamers (ethylene oxide propylene oxide block copolymers) of differing HLBs, lecithins, oleic acid and oleic acid salts, sorbitan esters (Span 20, 40, 60, 80 and 85), propylene glycol monostearate and monolaurate, glycerol monostearate and monooleate, Brij types (fatty alcohol-PEG ethers) of differing HLBs (for example, PEG 10 cetyl ether, PEG 20 oleyl ether, etc.), Myrj types (fatty acid-PEG esters) of differing HLBs (for example, PEG 40 monostearate; PEG 100 monostearate and the like), sodium dodecylsulphate (SDS), dioctyl sodium sulphosuccinate (DOSS), ethoxylated mono- and diglycerides of differing HLBs (Tagat types), sucrose fatty acid esters, fatty acid salts (Na, K, Ca, Mg, Al etc.), ethoxylated triglycerides (polyoxyethylated castor oil (40), polyoxyethylated hydrogenated castor oil (40 and 60), polyoxyethylated vegetable oils), sterols (cholesterol and wool wax alcohols) in concentrations of 0.001% to 20%, preferably 0.1% to 2%.

Lubricants used in the present invention include magnesium stearate, calcium stearate, calcium behenate, talc, colloidal silicic acid, stearic acid, precirol (mixture of mono-, di- and triesters of palmitic and stearic acid with glycerol), hydrogenated cottonseed oil, hydrogenated castor oil and polyethylene glycol of differing molecular weights.

The coated cores may additionally include a polishing layer. Suitable polishing agents include polyethylene glycols of differing molecular weight or mixtures thereof, talc, surfactants (Brij types, Myrj types, glycerol monostearate and poloxamers), fatty alcohols (stearyl alcohol, cetyl alcohol, lauryl alcohol and myristyl alcohol, and mixtures thereof).

The taste-masked pharmaceutical composition of the present invention may be packed in the form of a suspension in which the coated cores of ciprofloxacin are suspended in a suitable dispersion medium or as sachets comprising coated cores of ciprofloxacin.

An oily dispersion medium is used for suspending the coated cores of the present invention. Suitable oily dispersion media include almond oil, arachis oil, olive oil, poppy-seed oil, ground-nut oil, cottonseed oil, soyabean oil, maize oil, ethyl oleate, oleyl oleate, isopropyl myristate and isopropyl palmitate, medium chain glycerides. For example, medium chain triglycerides may be used.

The dispersion media may further include one or more pharmaceutically acceptable excipients such as emulsifiers, antioxidants, preservatives, colorants, sweeteners or flavorant.

The coated cores of ciprofloxacin and the dispersion medium may be packed separately. The suspension is prepared by the patient before use by adding the separately packed cores to the dispersion medium.

The taste-masked compositions of the present invention may be prepared according to a process that includes:

(i) dispersing drug and binder in a solvent to form a drug-dispersion;

(ii) spraying the dispersion of step (i) over the inert cores;

(iii) dispersing water-insoluble and water-soluble coating agent, and optionally a surfactant, in a solvent to form a coating dispersion; and

(iv) coating the cores of step (ii) with the coating dispersion of step (iii) to obtain microgranular cores comprising a taste-masking coating.

The solvent used in the preparation of the taste-masked composition of the present invention may include aqueous or non-aqueous solvents.

The present invention also relates to a method of treating bacterial infections, for example urinary tract infections, lower respiratory infections, anthrax, intra-abdominal infections, and skin and skin-structure infections, through administration of the taste-masked pharmaceutical composition of the present invention to a patient in need.

The following examples represent various embodiments according to the present invention. The examples are given solely for the purpose of illustration and are not to be construed as limitations of the present invention, as many variations thereof are possible without departing from the spirit and scope of the invention.

### EXAMPLES 1 AND 2

### Oral Suspension Comprising 5% and 10% W/W of Ciprofloxacin Respectively

| | | | |
|---|---|---|---|
| | | Example 1 | Example 2 |
| | | Quantity | Quantity |
| | | (Percentage | (Percentage |
| S. No. | Ingredients | w/w) | w/w) |

Core

| | | |
|---|---|---|
| Ciprofloxacin | 4.42 | 8.85 |
| Non-Pareil Seeds | 8.84 | 8.85 |
| Polyvinylpyrrolidone | 0.88 | 0.88 |
| Purified Water | q.s. | q.s. |

Taste Masked Coating

| | | |
|---|---|---|
| Neutral Copolymer of Ethyl | 3.08 | 4.04 |
| Acrylate and Methyl Methacrylate | | |
| Hydroxypropylmethylcellulose | 3.08 | 4.04 |
| PEG (20) Sorbitan Monolaurate | 0.04 | 0.05 |
| Magnesium Stearate | 0.88 | 1.16 |
| Purified Water | q.s. | q.s. |

Diluent

| | | |
|---|---|---|
| Miglyol | 54.84 | 50.21 |
| Sucrose | 21.94 | 20.08 |
| Lecithin | 0.78 | 0.72 |
| Colloidal Silicon Dioxide | 1.1 | 1.01 |
| Flavor | 0.12 | 0.11 |

Process:
(i) Polyvinylpyrrolidone was dissolved in purified water and ciprofloxacin was added to it to form a uniform dispersion.
(ii) The dispersion of step (i) was sprayed over a fluidized bed of non-pareil seeds.
(iii) The coating dispersion was prepared by dispersing neutral copolymer of ethyl acrylate and methyl methacrylate, hydroxypropylmethylcellulose, PEG (20) sorbitan monolaurate and magnesium stearate in purified water.
(iv) The drug layered microgranular cores of step (ii) were coated with a coating dispersion of step (iii).
(v) The coated microgranular cores were dried to achieve a water content between 6.0% to 8.0%.

### EXAMPLES 3 AND 4

### Oral Suspension Comprising 5% and 10% W/W of Ciprofloxacin Respectively

| | | | |
|---|---|---|---|
| | | Example 1 | Example 2 |
| | | Quantity | Quantity |
| | | (Percentage | (Percentage |
| S. No. | Ingredients | w/w) | w/w) |

Drug Layered Beads:

| | | |
|---|---|---|
| Ciprofloxacin | 5.50 | 11.90 |
| Sugar Spheres | 11.00 | 11.90 |
| Polyvinylpyrrolidone | 1.10 | 1.190 |
| Purified Water | q.s. | q.s. |

Sub-Coating

| | | |
|---|---|---|
| Hydroxypropylmethylcellulose | 3.00 | 4.25 |
| Polyethylene Glycol 400 | 0.176 | 0.250 |
| Talc | 0.352 | 0.500 |
| Purified Water | q.s. | q.s. |

Taste Masked Coating

| | | |
|---|---|---|
| Neutral Copolymer of Ethyl | 1.64 | 2.33 |
| Acrylate and Methyl | | |
| Methacrylate | | |
| Hydroxypropylmethylcellulose | 1.09 | 1.56 |
| PEG (20) Sorbitan Monolaurate | 0.032 | 0.045 |
| Magnesium Stearate | 0.396 | 0.562 |
| Purified Water | q.s. | q.s. |

Diluent

| | | |
|---|---|---|
| Caprylic/Capric Triglyceride | 70.65 | 70.65 |
| (Medium Chain Triglyceride) | | |
| Sucrose | 28.00 | 28.00 |
| Soy Lecithin | 1.00 | 1.00 |
| Colloidal Silicon Dioxide | 0.10 | 0.10 |
| Flavor | 0.25 | 0.25 |

Process:
(i) Polyvinylpyrrolidone was dissolved in purified water and ciprofloxacin was added to it to form a uniform dispersion.
(ii) The dispersion of step (i) was sprayed over a fluidized bed of sugar spheres.
(iii) The sub-coating dispersion was prepared by dispersing hydroxypropylmethylcellulose, polyethylene glycol 400 and talc in purified water.
(iv) The drug-layered microgranular cores of step (ii) were coated with the coating dispersion of step (iii).
(v) The coating dispersion was prepared by dispersing neutral copolymer of ethyl acrylate and methyl methacrylate, hydroxypropylmethylcellulose, PEG (20) sorbitan monolaurate and magnesium stearate in purified water.
(vi) The sub-coated microgranular cores of step (iv) were coated with the coating dispersion of step (v).
(vii) The coated microgranular cores were dried to achieve water content of between 6.0% to 8.0%.

### EXAMPLES 3 & 4

### LOD Values of Microgranular Cores of Ciprofloxacin

The LOD values of the microgranular cores of ciprofloxacin as per Examples 3 & 4 were determined under vacuum conditions at 120° C. for 1 hour.

TABLE 1

LOD (% w/w)

| Condition | Example 3 | Example 4 |
|---|---|---|
| Initial | 7.81 | 7.47 |
| 1M; 40° C./75% RH | 6.53 | 6.53 |
| 2M; 40° C./75% RH | 7.01 | 6.65 |
| 3M; 40° C./75% RH | 6.89 | 7.06 |

While several particular forms of the invention have been illustrated and described, it will be apparent to those skilled in the art that various modifications and combinations of the invention detailed in the text can be made without departing from the spirit and scope of the invention.

We claim:

1. A taste masked pharmaceutical composition comprising a microgranular core of ciprofloxacin and a taste-masking coating.

2. The taste masked pharmaceutical composition of claim 1, wherein the ciprofloxacin is present as the freebase form.

3. The taste masked pharmaceutical composition of claim 1, wherein the microgranular core of ciprofloxacin comprises an inert core layered with ciprofloxacin.

4. The taste masked pharmaceutical composition of claim 3, wherein the inert core comprises pharmaceutically acceptable inert insoluble materials, soluble materials, or commercially available products.

5. The taste masked pharmaceutical composition of claim 4, wherein the insoluble inert cores comprises one or more of dicalcium phosphate, microcrystalline cellulose.

6. The taste masked pharmaceutical composition of claim 4, wherein the soluble inert cores comprises one or more of glucose, mannitol, lactose, xylitol, dextrose, sucrose and mixtures thereof.

7. The taste masked pharmaceutical composition of claim 4, wherein the commercially available inert cores comprise one or more of sugar spheres, non-pareil seeds, celpheres and mixtures thereof.

8. The taste masked pharmaceutical composition of claim 1, wherein the taste masking coating comprises a mixture of water insoluble and water soluble polymers.

9. The taste masked pharmaceutical composition of claim 8, wherein the water insoluble polymer comprises one or more of acacia gum, acrylic acid polymers and copolymers (polyacrylamides, polyacryldextrans, polyalkyl cyanoacrylates, polymethyl methacrylates), agar-agar, agarose, albumin, alginic acid and alginates, carboxyvinyl polymers, cellulose derivatives, such as, cellulose acetate, polyamides (nylon 6-10, poly(adipyl-L-lysines, polyterephthalamides and poly(terephthaloyl-L-lysines)), poly-caprolactam, polydimethylsiloxane, polyesters, poly(ethylene-vinyl acetate), polyglycolic acid, polylactic acid and its copolymers, polyglutamic acid, polylysine, polystyrene, shellac, xanthan gum, anionic polymers of methacrylic acid and methacrylic acid esters.

10. The taste masked pharmaceutical composition of claim 8, wherein the water soluble polymer comprises one or more of hydroxypropylcellulose, hydroxypropylmethylcellulose, methylcellulose, sodium carboxymethylcellulose, dextran, dextrins, cyclodextrins, polyethylene glycols, polyvinyl alcohols, polyvinylpyrrolidones, starch and starch-hydrolysates, for example, modified types of starch (gelatinized starch, celutab, maltodextrins), sugars and sugar replacements, such as, mono-, di- and oligosaccharides, sucrose, fructose, lactose, dextrose, mannitol, sorbitol and xylitol and alginic acid and alginates, tragacanth, pectins, gum arabic and gelatin.

11. The taste masked pharmaceutical composition of claim 8, wherein the taste masking coating comprises a mixture of neutral methyl ester and/or ethyl ester compounds of polymethacrylic acid and hydroxypropylmethylcellulose.

12. The taste masked pharmaceutical composition of claim 8, wherein the taste masking coating layer further comprises one or more pharmaceutically acceptable inert excipients.

13. The tasted masked pharmaceutical composition of claim 12, wherein the one or more pharmaceutically acceptable inert excipients comprises plasticizers, lubricants, wetting agents, or colorants.

14. The taste masked pharmaceutical composition of claim 13, wherein the plasticizers comprise one or more of diethyl phthalate, acetyl tributylcitrate, glycerol, diethyl sebacate, dimethyl phthalate, dibutyl phthalate, tributyl citrate, butyl stearate, polyethylene glycols of different chain lengths, glycerol monostearate, triacetin, castor oil and other native and synthetic oils, triethyl citrate, acetyl triethylcitrate, 1,2-propylene glycol, acetylated fatty acid glycerides and polyoxyethylene-polyoxypropylene copolymers.

15. The taste masked pharmaceutical composition of claim 13, wherein the wetting agents comprise one or more of sodium lauryl sulphate (USP), polysorbate (20, 40, 60, 80, 65, 61, 85 and 21), poloxamers (ethylene oxide propylene oxide block copolymers) of differing HLBs, lecithins, oleic acid and oleic acid salts, sorbitan esters (Span 20, 40, 60, 80 and 85), propylene glycol monostearate and monolaurate, glycerol monostearate and monooleate, Brij types (fatty alcohol-PEG ethers) of differing HLBs (for example, PEG 10 cetyl ether, PEG 20 oleyl ether etc.), Myrj types (fatty acid-PEG esters) of differing HLBs (for example PEG 40 monostearate; PEG 100 monostearate and the like), sodium dodecylsulphate (SDS), dioctyl sodium sulphosuccinate (DOSS), ethoxylated mono- and diglycerides of differing HLBs (Tagat types), sucrose fatty acid esters, fatty acid salts (Na, K, Ca, Mg, Al etc.), ethoxylated triglycerides (polyoxyethylated castor oil (40), polyoxyethylated hydrogenated castor oil (40 and 60), polyoxyethylated vegetable oils), and sterols (cholesterol and wool wax alcohols).

16. The taste masked pharmaceutical composition of claim 13, wherein the lubricants comprise one or more of magnesium stearate, calcium stearate, calcium behenate, talc, colloidal silicic acid, stearic acid, precirol (mixture of mono-, di- and triesters of palmitic and stearic acid with glycerol), hydrogenated cottonseed oil, hydrogenated castor oil and polyethylene glycol of differing molecular weights.

17. The taste masked pharmaceutical composition of claim 1, wherein the composition provides a rapid release of ciprofloxacin at pH 1 and 4.5.

18. The taste masked pharmaceutical composition of claim 1, wherein the composition comprises more than 5% w/w of water.

19. A process for making a taste masked pharmaceutical composition, the process comprising:
(i) dispersing ciprofloxacin and binder in a solvent to form a drug-dispersion;
(ii) spraying the dispersion of step (i) over inert cores;
(iii) dispersing water-insoluble and water-soluble coating agents, and optionally a surfactant, in a solvent to form a coating dispersion;
(iv) coating the cores of step (ii) with the coating dispersion of step (iii) to obtain microgranular cores.
